(19) United States
(12) Patent Application Publication
(10) Pub. No.: US 2010/0256475 A1
(43) Pub. Date: Oct. 7, 2010

(54) PHYSIOLOGICAL-MEASUREMENT HEALTH-CARE SPORT SUIT

(76) Inventor: Cheng-Tang CHIANG, Taichung City (TW)

Correspondence Address: Muncy, Geissler, Olds & Lowe, PLLC, 4000 Legato Road, Suite 310, FAIRFAX, VA, 22033, US

(21) Appl. No.: 12/419,414

(22) Filed: Apr. 7, 2009

(51) Int. Cl. A61B 5/04; A61B 5/103

(52) U.S. Cl. 600/388; 600/595

(57) ABSTRACT

The present invention proposes a physiological-measurement health-care sport suit, which comprises two sensing members, an electric conduction coat, and a measurement device. Two pieces of electric conduction cloth are arranged on appropriate positions of the electric conduction coat and function as signal transmission media. The two sensing members respectively have a sensing electrode, and the sensing electrodes are respectively electrically connected to a piece of electric conduction cloth. The measurement device is arranged on the electric conduction coat and electrically connected to the two pieces of electric conduction cloth. When a user wears the suit having the two sensing members, the sensing electrodes respectively contact two test points on the user's body to obtain physiological potential signals. Then, the signals are transferred through the electric conduction cloth to the measurement device.

### FIELD OF THE INVENTION

The present invention relates to a measurement signal device of a physiological measurement system, particularly to a health-care sport suit capable of physiological measurement of a user.

### BACKGROUND OF THE INVENTION

An U.S. Pat. No. 6,945,940 disclosed a two-point physiological signal measurement device featuring larger contact areas between hands and electrodes, wherein the electrodes of sensors contact the hands of a user.

Refer to FIG. 7 and FIG. 8 for two-point physiological measurement suits disclosed in U.S. Pat. No. 6,755,795 and No. 7,211,053, which are characterized in that sensors 80 and 81 are respectively arranged on the breast region and the sleeve region of a suit. The sensors 80 and 81 cannot perform detection except they contact the user. Therefore, two girdles 82 and 83 are used to bind up the breast region and sleeve region of the suit and make the sensors 80 and 81 tightly contact the user. Thereby, the sensors 80 and 81 can detect physiological potential signals and transmit the signals to a microprocessor 84. Then, the microprocessor 84 analyzes the signals to obtain the physiological status of the user. However, the girdles 82 and 83 binding the breast and wrist of the user are likely to discomfort and inconvenience the user. Besides, the sensors 80 and 81 are fixedly installed on a suit, which not only makes the suit hard to clean or wash but also limits the application of the products, i.e. the sensors 80 and 81 cannot be installed on another suit. Thus is limited the market thereof.

Further, the abovementioned physiological signal measurement suits are unsuitable for sports having too great a movement because the user must always take notice of whether the sensors 80 and 81 tightly contact the body.

### SUMMARY OF THE INVENTION

The primary objective of the present invention is to provide a physiological-measurement health-care sport suit, wherein protection rings on the cuffs of the suit function as physiological potential signal measurement elements, and an electric conduction coat functions as the signal transmission medium, whereby the physiological monitoring system can effectively obtain measurement signals when the user wears the electric conduction coat.

To achieve the abovementioned objective, the present invention proposes a physiological-measurement health-care sport suit, which comprises two sensing members, an electric conduction coat, and a measurement device, wherein two sensing members respectively have a sensing electrode arranged on the inner surface of the sensing member. The sensing electrodes contact the user's body and obtain physiological potential signals. The electric conduction coat has two pieces of electric conduction cloth respectively electrically connected to the two sensing electrodes. The measurement device is arranged on the electric conduction coat and electrically connected to the two pieces of electric conduction cloth.

When the user wears the physiological-measurement health-care sport suit, the wrists of the user are respectively sleeved by the sensing member. The sensing electrodes of the sensing members contacts hands and have fine detection ability to obtain the physiological potential signals. Then, the physiological potential signals are transferred from the sensing electrodes through the electric conduction cloth to the measurement device.

### BRIEF DESCRIPTION OF THE DRAWINGS

FIG. 1 is a diagram schematically showing one structure of a physiological-measurement health-care sport suit according to the present invention;

FIG. 2 is a diagram schematically showing the structure of a button according to the present invention;

FIG. 3 is a diagram schematically showing another structure of a physiological-measurement health-care sport suit according to the present invention;

FIG. 4 is a diagram schematically showing a first embodiment of the present invention;

FIG. 5 is a diagram schematically showing a second embodiment of the present invention;

FIG. 6 is a block diagram schematically showing the architecture of a physiological-measurement health-care sport suit according to the present invention;

FIG. 7 is a diagram schematically showing an U.S. Pat. No. 6,755,795; and

FIG. 8 is a diagram schematically showing an U.S. Pat. No. 7,211,053.

### DETAILED DESCRIPTION OF THE PREFERRED EMBODIMENTS

Below, the embodiments are described in detail in cooperation with the drawings to make easily understood the objectives, characteristics, and efficacies of the present invention.

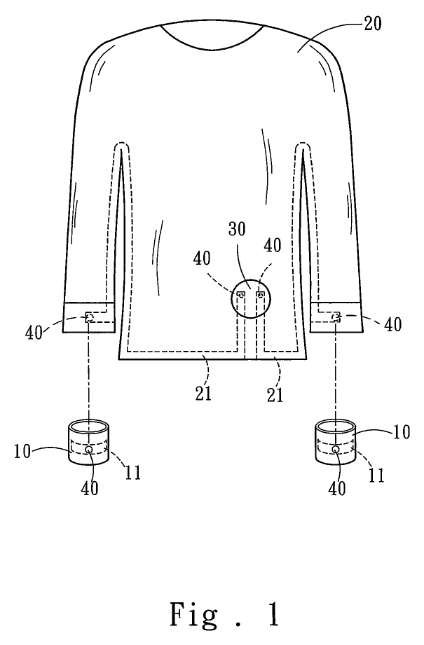

Refer to FIG. 1. The present invention proposes a physiological-measurement health-care sport suit, which comprises two sensing members 10, an electric conduction coat 20, and a measurement device 30. The two sensing members 10 may be in form of protection rings. The size of the protection rings can be adjusted to meet the user's wrists. The two sensing members 10 respectively have a sensing electrode 11 arranged on the inner surface of the sensing member 10. In the drawings, the electric conduction coat 20 is in form of a top wear. However, the present invention does not limit the electric conduction coat 20 to be a top wear. The electric conduction coat 20 has two pieces of electric conduction cloth 21 sewn on or stuck to the electric conduction coat 20. The electric conduction cloth 21 may be sewn on the electric conduction coat 20 along the seams of the electric conduction coat 20. The measurement device 30 is arranged on the electric conduction coat 20 and electrically connected to the two pieces of electric conduction cloth 21. The measurement device 30 is not limited to a specified position but may be arbitrarily arranged on a position suitable to the style or pattern of the coat.

Refer to FIG. 2. Buttons 40 which can be separated and transmit physiological potential signals are used to electrically connect the sensing electrodes 11 and the electric conduction cloth 21 and also used to electrically connect the measurement device 30 and the electric conduction cloth 21. Each button 40 has two components respectively arranged on the two elements to be joined by the button 40. As the two components of the button 40 can be separated, the sensing members 10 and the measurement device 30 can be detached from the electric conduction coat 20 to convenience washing the electric conduction coat 20.

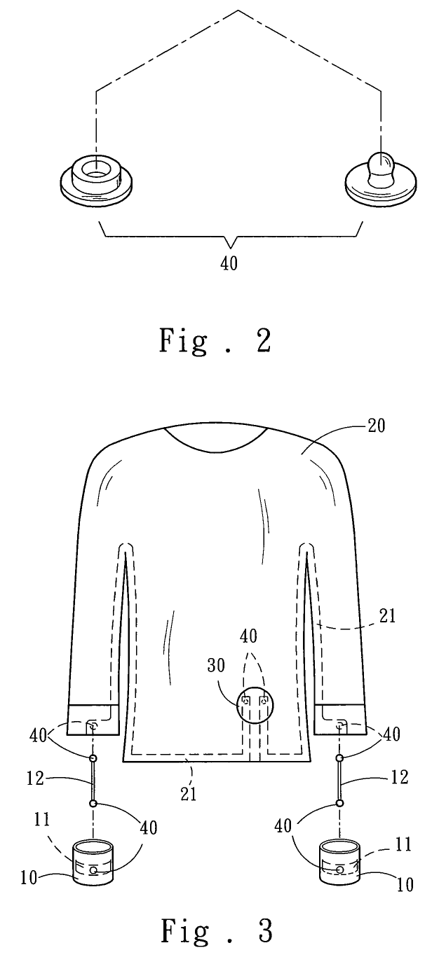

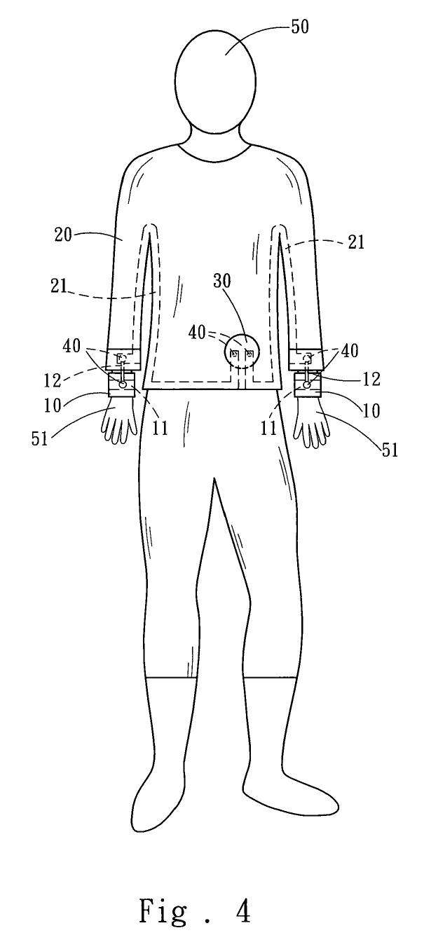

Refer to FIG. 3. The two sensing members 10 may respectively have a signal transmission cable 12. One terminal of each signal transmission cable 12 is electrically connected to one sensing electrode 11 via one button 40, and the other terminal of the signal transmission cable 12 is electrically connected to one piece of electric conduction cloth 21 via another button 40. Thereby, the sensing members 10 and the electric conduction coat 20 have appropriate mobility space, and the user wearing the electric conduction coat 20 may feel more comfortable.

Refer to FIG. 4 for a preferred embodiment of the present invention. In this embodiment, the electric conduction coat 20 is in form of a top wear, and the electric conduction cloth 21 is sewn on the electric conduction coat 20 along the seams and edges of the electric conduction coat 20. The electric conduction cloth 21 extends to the cuffs. Two wrists 51 of a user 50 are respectively sleeved by a two sensing member 10. The signal transmission cable 12 and the button 40 electrically connect the sensing electrode 11 and the electric conduction cloth 21. The sensing electrodes 11 obtain physiological potential signals from the wrists 51. The electric conduction cloth 21 of the electric conduction coat 20 functions as the medium to transmit the signals to the measurement device 30. The measurement device 30 receives and calculates the signals to obtain the physiological status of the user 50.

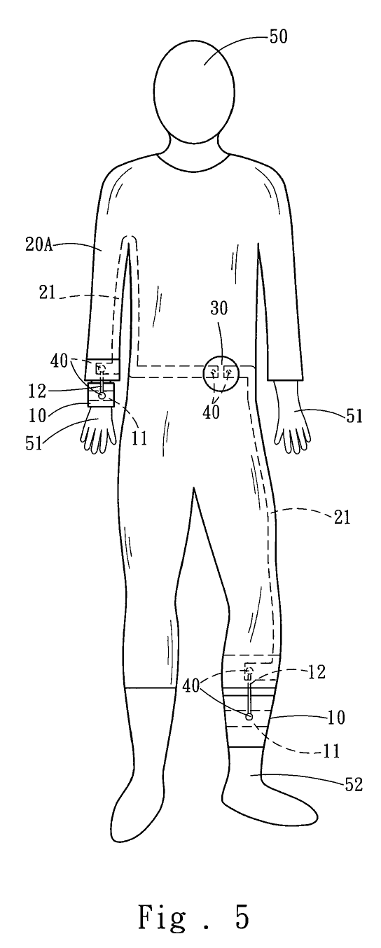

Refer to FIG. 5 for another preferred embodiment of the present invention. In this embodiment, the electric conduction coat 20A is in form of a suit having a top wear and a pair of trousers, and the electric conduction cloth 21 extends to the cuff and the bottom of a trouser leg. The wrist 51 and the ankle 52 of a user 50 are respectively sleeved by the sensing member 10. Thus, the measurement device 30 receives and calculates the physiological potential signals to obtain the physiological status of the related regions of the user 50.

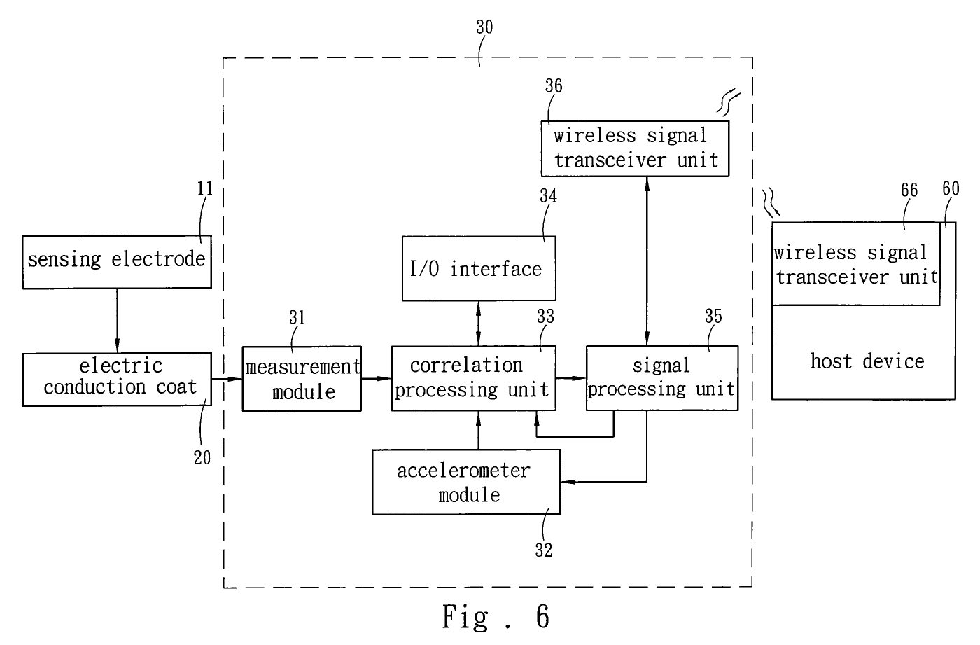

Refer to FIG. 6. The measurement device 30 includes a measurement module 31, an accelerometer module 32, a correlation processing unit 33, an I/O interface 34, a signal processing unit 35, and a wireless signal transceiver unit 36. The parametric feedback signals of the measurement module 31, the correlation processing unit 33 and the signal processing unit 35 are used to calculate the physiological signals of the user 50. Thus, the physiological signals of the user 50 are obtained via a two-point measurement.

Further, the parametric feedback signals of the accelerometer module 32, the correlation processing unit 33 and the signal processing unit 35 are used to calculate the movement status of the measurement device 30. Thus, in addition to the physiological signals of the user 50, the present invention also obtains the movement status of the user 50 from the accelerometer module 32 to determine the posture of the user 50, such as lying, standing, stillness, or moving. Such a function can expand the application of the present invention.

The physiological-measurement health-care sport suit may further comprise a host device 60, which may be in form of a measurement signal receiver or a physiological monitoring system. The host device 60 may be hung on the user 50 or installed in a home-care system, a sport apparatus, or a communication device. The host device 60 may include a wireless signal transceiver unit 66, which is wirelessly linked to the wireless signal transceiver unit 36 of the measurement device 30, whereby the host device 60 can receive the measurement results of the measurement module 31 and the accelerometer module 32. Then, the host device 60 takes appropriate actions according to the measurement results.

The present invention can apply to elders or patients. The present invention can cooperate with a home-care system or a communication device. The host device 60 can obtain the movement status of the user, such as lying, standing, stillness or moving from the parametric feedback signals of the accelerometer module 32, the correlation processing unit 33 and the signal processing unit 35 of the measurement device 30. The host device 60 can also learn the physiological status of the user in real time from the measurement module 31. Therefore, the present invention has the functions of health caring and home nursing.

In the physiological-measurement health-care sport suit of the present invention, the sensing electrodes 11 obtain the physiological potential signals, and the signals is transferred to the measurement device 30 via the buttons 40 and the electric conduction cloth 21 of the electric conduction coat 20 or 20A. In comparison with the conventional technologies, the present invention has the following advantages:

1. As the sensing members 10 are in form of protection rings, which sleeve and tightly contact the wrists 51 of the user, the user needn't worry about incorrect measurement caused by imperfect contact even when the body has a greater movement.

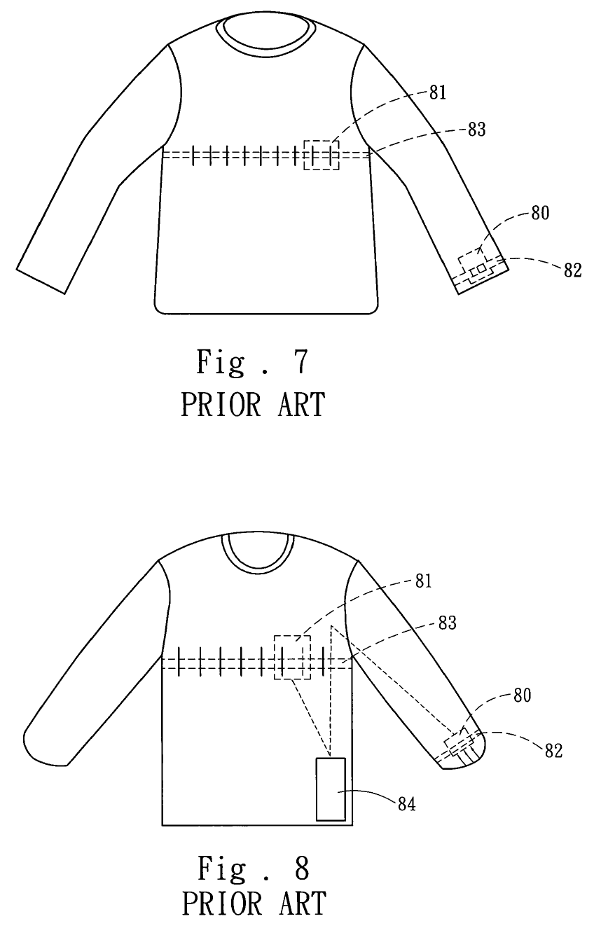

2. As the sensing members 10 are fabricated to have a form of protection rings sleeving and tightly contacting the wrists 51 of the user, the present invention is exempt from the conventional uncomfortable girdles 82 and 83 (as shown in FIGS. 7 and 8).

3. As the sensing members 10 and the measurement device 30 can be easily attached to and detached from the electric conduction coats 20 and 20A via the buttons 40, the cleaning and washing of the electric conduction coats 20 and 20A is convenient.

4. As the sensing members 10 and the measurement device 30 can be easily attached to and detached from the electric conduction coats 20 and 20A via the buttons 40, the application of the components of the present invention is not limited to a specified electric conduction coat 20 or 20A. Thus is expanded the application of the present invention.

What is claimed is:

1. A physiological-measurement health-care sport suit comprising
two sensing members each having a sensing electrode arranged on an inner surface of said sensing member;
an electric conduction coat having two pieces of electric conduction cloth respectively electrically connected to said sensing electrode of said sensing member; and
a measurement device arranged on said electric conduction coat and electrically connected to two pieces of said electric conduction cloth.

2. The physiological-measurement health-care sport suit according to claim 1, wherein buttons, which are separable and able to transmit physiological potential signals, are used to electrically connect said sensing electrodes and said electric conduction cloth and also used to electrically connect said measurement device and said electric conduction cloth.

3. The physiological-measurement health-care sport suit according to claim 2, wherein said sensing members respectively have a signal transmission cable; one terminal of said signal transmission cable is electrically connected to said sensing electrode via said button, and the other terminal of said signal transmission cable is electrically connected to one piece of said electric conduction cloth via another said button.

4. The physiological-measurement health-care sport suit according to claim 1, wherein said electric conduction coat is in form of a top wear; when a user wears said physiological-measurement health-care sport suit, two wrists of said user are respectively sleeved by said sensing member.

5. The physiological-measurement health-care sport suit according to claim 1, wherein said electric conduction coat is in form of a suit having a top wear and a pair of trousers; when a user wears said physiological-measurement health-care sport suit, a wrist and an ankle of said user are respectively sleeved by said sensing member.

6. The physiological-measurement health-care sport suit according to claim 1, wherein said measurement device includes a measurement module measuring physiological signals of a user and an accelerometer module detecting a movement status of said measurement device.

7. The physiological-measurement health-care sport suit according to claim 6 further comprising a host device receiving measurement results of said measurement module and said accelerometer module of said measurement device.

8. The physiological-measurement health-care sport suit according to claim 7, wherein said host device has a wireless signal transceiver unit, and said measurement device has a wireless signal transceiver unit; said host device and said measurement device are wirelessly linked by said wireless signal transceiver units.

9. The physiological-measurement health-care sport suit according to claim 7, wherein said measurement device is hung on said user's body.

10. The physiological-measurement health-care sport suit according to claim 1, wherein said electric conduction cloth is stuck to or sewn on an appropriate position of said electric conduction coat.
